SEQUENCE LISTING (1 sequence)

SEQ ID NO: 1 - RNA, 22 nt, synthetic construct
  source 1..22: mol_type = other RNA
  modified_base 1: mod_base = OTHER; note = uracil
  modified_base 15: mod_base = OTHER; note = uracil
  modified_base 12..13: mod_base = OTHER; note = uracil
  modified_base 21: mod_base = OTHER; note = uracil
  modified_base 3: mod_base = OTHER; note = uracil
tctcccaacccttgtaccagtg

(19) United States
(12) Patent Application Publication
(10) Pub. No.: US 2025/0345399 A1
(43) Pub. Date: Nov. 13, 2025

(54) THROMBOMODULIN PROTEIN AND MIRNA FOR PREVENTING OR TREATING OSTEOATHRITIS

(71) Applicant: Kaohsiung Medical University, Kaohsiung City (TW)

(72) Inventors: Tsung-Lin Cheng, Tainan City (TW); Chung-Hwan Chen, Tainan City (TW); Je-Ken Chang, Kaohsiung City (TW)

(73) Assignee: Kaohsiung Medical University, Kaohsiung City, TW (TW)

(21) Appl. No.: 18/656,608

(22) Filed: May 7, 2024

(51) Int. Cl. A61K 38/36; A61P 19/02; C12N 15/113

(52) U.S. Cl. CPC A61K 38/366; A61P 19/02; C12N 15/113; C12N 2310/113; C12N 2320/35

(57) ABSTRACT

A method for optimizing osteoarthritis (OA) treatment by promoting chondrocyte cell proliferation and migration using thrombomodulin (TM) or soluble TM. This method involves blocking interleukin 1β (IL-1β)-mediated signaling to prevent the loss of bone integrity and maintain knee joint function. Additionally, the administration of miR-up-TM enhances the expression of TM protein, thereby protecting against cartilage damage and potentially preventing cartilage-related diseases.

This application also contains a Sequence Listing in a computer readable form, the file name is 4214-KMU-SequenceListing, created on Mar. 29, 2024, the size is 5 KB, which is incorporated herein by reference. The sequence information contained in the Docx file and PDF file is identical to the sequence information contained in the computer readable form (XML File).

### FIELD OF THE INVENTION

The present invention relates to the method of preventing or treating arthritis, especially osteoarthritis.

### BACKGROUND OF THE INVENTION

Osteoarthritis (OA), the most common joint disease, affected approximately 303 million people globally in 2017. The major feature of OA is the destruction of articular cartilage, often accompanied by synovial inflammation, joint capsule hypertrophy, osteophyte formation, and subchondral bone thickening. The pathogenesis of OA is complex and multifactorial. Chondrocytes play a central role in controlling articular cartilage structure and function, thus regulating the turnover of extracellular matrix components, including collagen, glycoproteins, proteoglycans, and hyaluronan, in maintaining tissue homeostasis. Despite increased knowledge of the function of chondrocytes and the underlying pathology associated with OA and chondro-cartilage disorders, disease-improving treatments, and preventative strategies for patients with OA are lacking. Therefore, novel, affordable, and practical treatment approaches for OA are urgently required.

Thrombomodulin (TM), known as CD141, is a type-I transmembrane glycoprotein expressed by several cell types, including chondrocytes, osteoblasts, endothelial cells, monocytes/macrophages, and keratinocytes. The TM has five structural domains from the N- to the C-terminus: domain 1 (TMD1), a C-type lectin-like domain; domain 2 (TMD2), containing six epidermal growth factor (EGF)-like structures; domain 3 (TMD3), a serine/threonine-rich domain; domain 4 (TMD4), the transmembrane domain; and domain 5 (TMD5), the cytoplasmic domain. These different domains of TM exhibit distinct properties and thus participate in several processes, including protecting against excess inflammation, coagulation, and fibrinolysis; reducing bone loss; and promoting bone repair, cutaneous wound healing, cell proliferation, and cell-cell adhesion. In addition to being an integral membrane protein, soluble forms of TM (sTM) exist, which comprise various extracellular domains. sTM may be generated in part by cleavage by rhomboid-like-2 membrane protease (RHBDL2).

Protein expression of TM is regulated via transcriptional and post-transcriptional mechanisms. For example, in endothelial cells, TM is increased by the transcription factor Krüppel-like factor 2 (KLF2), which is upregulated via inhibition of miR-150. Besides, inhibition of miR-150 protects chondrogenic cells ATDC5 against cytokine (IL-1)-induced injury. The previous studies indicate that TM, expressed by osteoclasts and osteoblasts, inhibits bone loss and promotes bone healing. However, its specific role in chondrocytes, cartilage homeostasis, and articular cartilage-related disorders remains limited.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1H show the shedding of the TM extracellular domain by RHBDL2 contributes to cell proliferation and migration in chondrocytes through its EGF-like domain. FIG. 1A shows the human chondrocytes (TC28a2) incubated under serum starvation. Then, the cell lysates were collected at indicated time points for western blotting to evaluate the TM protein level. Actin was used as an internal loading control. FIG. 1B shows the concentrated conditioned mediums (CMs) from FIG. 1A used to evaluate the protein level of soluble TM (sTM). Glutathione S-transferase (GST) was added as an internal control. FIG. 1C evaluates sTM protein levels in serum-free CM harvested with DCI (RHBDL2 inhibitor) after 48 h of incubation of human chondrocytes. FIG. 1D shows the WST-1 cell proliferation assay to evaluate the effect of recombinant TMD2-3 (rTMD23; TMD2 is an EGF-like repeat domain) on chondrocytes after 48 hours of treatment. FIG. 1E shows the growth curve of TM shRNA (shTM)-transfected TC28a2 cells in two days. FIG. 1F shows the growth curve of shTM-transfected TC28a2 cells in two days. FIG. 1G evaluated the effect of rTMD23 on shTM chondrocytes by MTT cell proliferation assay. FIG. 1H shows the transwell cell migration assay to evaluate the effect of rTMD23 on shTM chondrocytes. Scale bar: 100 μm. * P<0.05; ** P<0.01; *** P<0.001. All experiments were repeated at least three times.

FIG. 2A-2H show the rTMD123 treatment attenuates IL-1β-reduced TM and sTM protein levels, chondrocyte proliferation, and migration by reducing STAT3 signaling and MMP13 expression. FIG. 2A and FIG. 2B respectively show that after human chondrocytes (TC28a2) were treated with IL-1β for 24 h, cell lysates were collected, and conditioned medium was used to evaluate TM protein levels and sTM production, GADPH and GST was respectively used as an internal control. FIG. 2C shows the effect of IL-1β on chondrocytes using WST-1 cell proliferation assay after treatment for 48 hours. FIG. 2D shows that rTMD123 reversed IL-1β-reduced cell proliferation in a dose-dependent manner. FIG. 2E shows the use of transwell cell migration assay to evaluate the effect of rTMD123 on IL-1β-inhibited cell migration. FIG. 2F-2H show that human chondrocytes treated with rTMD123 can inhibit the expression of IL-1β-mediated STAT3 signaling and MMP13. Scale bar: 100 μm.* P<0.05; ** P<0.01; *** P<0.001.

FIG. 3A-3H show the rTMD123 treatment protects knee functions in ACLT-induced OA mice by increasing articular cartilage TM expression and reducing MMP 13 level. FIG. 3A illustrates the experimental design. The arrowhead indicates the ACLT surgery was performed. The “+” indicates the injection of rTMD123. The black arrows indicate mice undergoing tests to assess knee function. FIG. 3B show the results of weight-bearing distribution test. FIG. 3C show the results of treadmill test at week 4. N=5 in each group. FIG. 3D shows the distribution of TM in IHC stained knee joints samples of mice taken out at 4 weeks post-sacrifice after ACLT. FIG. 3E shows the distribution of MMP13 in IHC stained samples of mouse knee joints. The red regions indicate the area where MMP13 is distributed. FIG. 3F shows western blot analysis of mouse knee joints to assess the expression of TM and MMP13. The red-brown color represents signal-positive cells. FIG. 3H shows the quantitative analysis of MMP13 in mice knee joints with different treatments. Scale bar: 50 μm. * P<0.05; ** P<0.01; *** P<0.001.

FIG. 4A-4H show the accelerated knee joint function deterioration due to deletion of TM lectin-like domain after ACLT surgery improved by rTMD123. FIG. 4A is a graph showing the difference of TM protein structure between TM^{wt/wt} and TM^{LeD/LeD}. FIG. 4B shows the results of the weight-bearing distribution test of TM^{wt/wt} and TM^{LeD/LeD} mice at two weeks after ACLT. FIG. 4C shows the results of treadmill testing in TM^{wt/wt} and TM^{LeD/LeD} mice at two weeks after ACLT. FIG. 4D shows the results of the weight-bearing distribution test after mice treated with 100 μg/kg rTMD123 for 4 weeks after ACLT. FIG. 4E shows the results of treadmill testing in mice treated with 100 μg/kg rTMD123 for 4 weeks after ACLT. FIG. 4F is a cartilage distribution diagram of stained sections of the knee joint of ACLT-operated mice, stained with safranin O staining and toluidine blue respectively, where red or dark blue areas represent chondrocytes. FIG. 4G shows the analysis of cartilage area from safranin O-stained sections of the knee joint in ACLT-operated mice. FIG. 4H is OARSI score of cartilage area from safranin O-stained sections of the knee joint in ACLT-operated mice. Scale bar: 50 μm. ** P<0.01; *** P<0.001.

FIG. 5A-5E show the delayed administration of rTMD123 one week after ACLT surgery still protected knee function in mice. FIG. 5A illustrates the experimental design. The arrowhead indicates the ACLT surgery was performed. The “+” indicates the injection of rTMD123. The black arrows indicate mice undergoing tests to assess knee function. FIG. 5B show the results of weight-bearing distribution test. FIG. 5C show the results of treadmill test. FIG. 5D is a quantitative analysis diagram of safranin-stained sections of knee joints of mice after surgery. FIG. 5E is a OARSI score of safranin-stained sections of knee joints of mice after surgery. Scale bar: 50 μm. * P<0.05; P<0.01; *** P<0.001.

FIG. 6A-6L show the miR-up-TM-enhanced KLF2/TM signaling is critical to protect knee functions by promoting chondrocyte growth and reducing MMP 13 level. FIG. 6A shows the analysis of cell lysates of chondrocytes (TC28a2) after transfection with a negative control miRNA (NC) at 20 nM and miR-up-TM at 5-20 nM for 24 hours by Western blot. FIG. 6B shows the quantitative analysis of KLF2 in chondrocytes treated with miR-up-TM. FIG. 6C shows the quantitative analysis of TM in chondrocytes treated with miR-up-TM. FIG. 6D shows that shTM abrogates miR-up-TM-enhanced TM levels using western blot analysis. FIG. 6E shows that shTM inhibits the growth of chondrocytes boosted by miR-up-TM. FIG. 6F shows that shTM abolished the effect of miR-up-TM on IL-1β-suppressed chondrocyte survival in mice after two days of treatment. FIG. 6G shows the results of the weight-bearing distribution test of mice treated with miR-up-TM+ shTM at the 4th week after surgery. FIG. 6H shows the results of the treadmill test of mice treated with miR-up-TM+shTM at the 4th week after surgery. FIG. 6I shows the expression distribution of TM and MMP13 in the stained sections of the knee joint treated with miR-up-TM+shTM. Red or red-brown color represents the protein expression signal of TM or MMP13, and blue is the nucleus. FIG. 6J shows western blot analysis of chondrocytes treated with miR-up-TM+shTM. FIG. 6K shows the TM quantitative analysis of chondrocytes treated with miR-up-TM+shTM. FIG. 6L shows the MMP13 quantitative analysis of chondrocytes treated with miR-up-TM+shTM. Scale bar: 50 μm. * P<0.05; ** P<0.01; *** P<0.001.

FIG. 7A-7B show the schematic diagram shows the role of the TM in chondrocytes and OA. FIG. 7A shows that excessive inflammatory factors, such as IL-1β, impede the expression of TM in chondrocyte and RHBDL2-mediated sTM production, leading to elevated STAT3/MMP13 signaling pathways and reduced chondrocyte growth and migration, ultimately leading to joint Cartilage damage and OA. FIG. 7B shows that miR-up-TM (miR-150 antagomir) enhances the expression of TM and the release of sTM in chondrocytes by increasing the KLF2 transcription factor, thereby improving the anti-inflammation and cell activity of chondrocytes, eventually leading to anti-OA effects.

### DETAILED DESCRIPTION OF THE INVENTION

miR-up-TM is a miRNA preparation that can enhance the expression of thrombomodulin (TM) in chondrocytes. The protein fragment of TM (rTMD123) has the function of assisting chondrocytes in resisting inflammatory factors and promoting cell growth and migration.

In both cell or murine osteoarthritis (OA) models, the results of the present invention demonstrate that individual administration of miR-up-TM and rTMD123 can effectively achieve anti-inflammatory effects, promote cell growth and migration, help maintain the integrity of articular cartilage, and enhance individual mobility, contributing to the prevention and treatment of OA.

The present invention prevents or treats OA through the following features:

(1) Using miR-up-TM to enhance the expression of TM in chondrocytes, or directly administering TM protein preparations can improve cell anti-inflammatory ability, cell proliferation and cell migration, thereby assisting cells in combating OA.

(2) TM is a native human protein with a highly conserved amino acid sequences, making it less likely to raise allergy concerns.

(3) miR-up-TM has the advantages of cost reduction and reduced administration frequency.

The present invention provides a method for the prevention or treatment of a cartilage-related disorder in a subject, comprising administering to the subject an effective amount of a composition comprising Thrombomodulin.

In one embodiment, wherein the composition further comprises a pharmaceutically acceptable carrier comprising plasma, optionally enriched with platelets, serum, water for injection, physiological saline, hyaluronan, chemically modified hyaluronan, saline, phosphate buffered saline, chondroitin sulfate, glucosamine, mannosamine, proteoglycan, proteoglycan fragments, chitin, chitosan, or a combination of two or more thereof.

In one embodiment, the cartilage-related disorder is osteoarthritis, degenerative joint disease, osteochondritis dissecans, rheumatoid arthritis, articular cartilage damage, achondroplasia, or cartilage defects.

In one embodiment, the composition reduces cellular inflammatory response, promotes cell growth and migration, increases the expression of Krüppel-Like Factor 2 (KLF2) and thrombomodulin, blocks interleukin-1β (IL-1)-mediated signal transduction, protects knee joints, prevents cartilage loss or damage, maintains the integrity of bone joints, and preserves the subject's mobility by administering the effective amount to the subject, thereby preventing or treating cartilage-related disorders.

In another embodiment, the IL-1β-mediated signaling comprises STAT3 and MMP13.

In one embodiment, the effective amount of Thrombomodulin is 1˜100 μg/kg. In a prefer embodiment, the effective amount of Thrombomodulin is 1˜10 μg/kg.

In one embodiment, the Thrombomodulin is natural or synthetic protein.

The present invention provides a method for the prevention or treatment of a cartilage-related disorder in a subject, comprising administering to the subject an effective amount of a composition comprising miR-up-TM.

In one embodiment, the composition further comprises a pharmaceutically acceptable carrier comprising plasma, optionally enriched with platelets, serum, water for injection, physiological saline, hyaluronan, chemically modified hyaluronan, saline, phosphate buffered saline, chondroitin sulfate, glucosamine, mannosamine, proteoglycan, proteoglycan fragments, chitin, chitosan, or a combination of two or more thereof.

In one embodiment, the cartilage-related disorder is osteoarthritis, degenerative joint disease, osteochondritis dissecans, rheumatoid arthritis, articular cartilage damage, achondroplasia, or cartilage defects.

In one embodiment, the composition reduces cellular inflammatory response, promotes cell growth and migration, increases the expression of Krüppel-Like Factor 2 (KLF2) and thrombomodulin, blocks interleukin-1β (IL-1B)-mediated signal transduction, protects knee joints, prevents cartilage loss or damage, maintains the integrity of bone joints, and preserves the subject's mobility by administering the effective amount to the subject, thereby preventing or treating cartilage-related disorders.

In another embodiment, the IL-1β-mediated signaling comprises STAT3 and MMP13.

In one embodiment, the effective amount of the miR-up-TM is 0.1˜20 nM. In a prefer embodiment, the effective amount of the miR-up-TM is 0.25˜10 nM. In a more prefer embodiment, the effective amount of the miR-up-TM is 0.5˜2 nM.

In another embodiment, the miR-up-TM increasing the expression of Thrombomodulin.

In another embodiment, the miR-up-TM is miR-150 antogomir.

In another embodiment, the miR-150 antogomir is RNA sequence of SEQ ID NO:1.

### Example

The following is a description of the method of the present invention. Although reference is made in this description to certain specific embodiments, this is provided for exemplary purposes only and should not be construed as limiting the invention to these specific embodiments. On the contrary, the spirit and scope of the invention are intended to include alternatives, modifications, and equivalents. Accordingly, the description and drawings should be regarded as illustrative rather than restrictive.

Numerous implementation details are provided in the specification to provide a thorough understanding of the present invention. However, one skilled in the art may be able to practice the present invention without these specific details. In some instances, methods, procedures, and materials that are already well known to those skilled in the art have not been described in detail to avoid obscuring essential features of the present invention.

### Sources of Antibodies and Reagents

Antibodies recognizing human TM (sc-13164), mouse TM (sc-7097), and glutathione S-transferase GST (sc-138) were obtained from Santa Cruz Biotechnology (Santa Cruz, CA, USA). The following antibodies were purchased from Cell Signaling Technology (Danvers, MA, USA): anti-STAT3 (9139) and anti-p-STAT3 (Tyr705, 9145). Antibodies against GAPDH (ab8245) and MMP13 (ab39012 and ab237604) were purchased from Abcam (Cambridge, UK). The recombinant GST protein (ab70456) was purchased from Abcam (Cambridge, MA, USA). The RHBDL2 serine protease inhibitor (3,4-dichloroisocoumarin [DCI]) and IL-1β (H6291) were purchased from Sigma-Aldrich (St. Louis, MO, USA). Lipofectamine-3000, mir Vana® miRNA inhibitor (antagomir) of miR-150 (#MH10070), and the corresponding negative control miRNA were purchased from Thermo Fisher Scientific.

### Experimental Animals

10 weeks old mice lacking the TM lectin-like domain (TM^{LeD/LeD}), and corresponding wild-type C57BL/6 mice (TM^{wt/wt}) were used. The animal care and experimental procedures were approved by the Institutional Animal Care and User Committee of Kaohsiung Medical University, Kaohsiung, Taiwan (Approval no: KMU-IACUC-109089).

### Statistical Analysis

Continuous data are expressed as mean±SD. The Student's t-test or Mann-Whitney U test was used to determine the significance of comparisons between the two groups. One-way ANOVA followed by post-hoc analysis (Tukey's test) was used for comparisons between more than two groups. P<0.05 was considered statistically significant.

### Embodiment 1: Expression of Recombinant TM Domains

The pCR3-EK vectors (Invitrogen) were used to express recombinant TM functional domains for purification and detection in human embryonic kidney 293 mammalian protein expression systems. Expressed recombinant proteins were applied to a nickel-chelating Sepharose column (Amersham Pharmacia Biotech). Next, recombinant TM domain-containing fractions were eluted, and purified fractions were pooled for use. Purified rTMD123 were examined by Coomassie blue staining and western blotting after gel electrophoresis.

### Embodiment 2: Cell Culture

The human articular chondrocyte cell line (TC28a2) was obtained from the American Type Culture Collection and cultured in Dulbecco's modified Eagle's medium (DMEM) containing 10% fetal bovine serum (FBS). Cells were cultured in a humidified atmosphere at 37° C. and 5% CO₂. Confluent cells were cultured in six-well plates under serum-free conditions. After incubation with RHBDL2 inhibitor (DCI), cell-free conditioned media (CMs) were collected with the addition of 20 μg/sample glutathione S-transferase (GST) as an internal control and then concentrated using Centricon tubes with a 10-kDa molecular weight cutoff (Amicon). The concentrated samples and cell lysates were then separated using SDS-PAGE and subsequently analyzed using western blotting. To generate TC28a2 cells that did not express TM (referred to as TM-silenced cells), the pSM2c vector system (GenDiscovery Biotechnology) expressing short hairpin RNA (shRNA) against TM (shTM) was transfected.

### Embodiment 3: Western Blotting

After SDS-PAGE, samples were transferred onto PVDF membranes (Millipore Sigma), blocked with 3% BSA-TBST (50 mM Tris-HCl, 150 mM NaCl, Tween-20; Millipore Sigma), and then probed with western blotting. Signals were detected using an enhanced chemiluminescence reagent (Amersham Pharmacia Biotech) using the LAS3000 imaging system (Fujifilm). ImageJ software was used to quantify the band intensities.

### Embodiment 4: Cell Proliferation/Viability Assay

Cells were seeded into 24-well plates at 600 μL, 3×10³ cells/well and incubated with recombinant rTMD123 at 37° C. in a 5% CO₂ atmosphere. The medium was replaced every two days. Cell proliferation and viability were quantified using an assay kit (WST-1, K301-500, Bio Vision; MTT, ab232855, Abcam) according to the manufacturer's instructions, and the absorbance of acetic acid-stopped reactions was measured at 450 nm or 590 nm (SPECTRAmax 340).

### Embodiment 5: Transwell Cell Migration Assay

Cell migration was evaluated using a 24-well chemotaxis chamber with a membrane of 8-μm pore size (Transwell). A cell suspension with 5×10⁴ cells/100 μL serum-free medium was added to the upper chamber, and 600 μL rTMD123 in serum-free medium was added to the lower chamber. Thereafter, the chambers were incubated at 37° C. for 12-24 h. Cells that did not migrate were wiped off the membrane using a cotton swab. The filter was developed using Liu's stain kit, and the number of remaining cells were counted by direct visualization under a light microscope.

### Embodiment 6: Anterior Cruciate Ligament Transection (ACLT)-Induced Knee OA

10 weeks old and 18-22 g male mice were randomly divided into two groups: ACLT and ACLT+rTMD123 groups (n=5/group). A total of about 50 mice were used. Under general anesthesia, both hind limbs were shaved and prepared for surgery under sterile conditions. OA was induced in the right knee in test group (ACLT and ACLT+rTMD123 groups), whereas a sham operation was performed on the left knee (single cutaneous incision and stitching) in control group. In the ACLT+rTMD123 group, 5 μL of rTMD123 dissolved in phosphate-buffered saline was injected into the right knee joints of mice through a microsyringe with a 34G needle. The injection was once a week for four weeks, with a total injection of 20 μL. The mice were then sacrificed through overdose of anesthesia, and the knees of the mice were surgically removed and histologically analyzed. The indicated rTMD123 is a liposome encapsulating miRNA inhibitor.

### Embodiment 7: Weight-Bearing Distribution Test

The effect of joint damage on weight distribution in the knees of mice was measured using a dual-channel weight averager, which independently quantifies the weight-bearing ability of each hind paw. Changes in hind paw weight distribution between OA and contralateral control limbs were used as an index of joint discomfort in the OA knee. Mice were placed in an angled Plexiglas chamber positioned such that each hind paw rested on a separate force plate. The force exerted by each hind limb was averaged over a 5-second period, and each data point was the mean of three 5-second readings. The change in hind paw weight distribution was calculated by determining the percentage difference in the weight between the left and right limbs. The weight-bearing tests were performed before ACLT surgery and each subsequent week until the mice were euthanized.

### Embodiment 8: Treadmill Test

Mice were habituated to run on a Columbus Instruments rodent treadmill, with training sessions performed before the ACLT surgery for 15 min/day at a speed of 10-15 m/min for one week. After the adaptation period, treadmill tests were performed twice weekly, and the data were averaged after treatment. All the mice were evaluated using an exercise program that consisted of a speed of 40 m/min. The recording time for running endurance was limited to 15 min and running stopped at the maximum duration of running endurance.

### Embodiment 9: Histology and Immunohistochemistry

The isolated proximal tibiae were fixed in 10% neutral buffered formalin and decalcified in 10% formic acid after euthanasia. Subsequently, 5-μm microsections were prepared in the coronary plane, stained with glycosaminoglycan with safranin O-Fast Green or toluidine, and quantified with Image-Pro Plus 5.0 software (Media Cybernetics). The density of the red-stained area relative to the total area (density/total area) in each group was calculated. The results of the histological study were assessed using microscopic scoring as recommended by the Osteoarthritis Research Society International (OARSI).

The safranin O-Fast Green is counterstained with 1% safranin O and 0.75% hematoxylin, and then stained with 1% Fast Green.

For immunohistochemical staining, endogenous peroxidase in the tissues was blocked with 3% hydrogen peroxide, and the samples were digested with enzymes for epitope retrieval. Thereafter, the sections were blocked with FBS for 1 h and incubated with primary antibodies against TM and MMP13 at 37° C. for 4 h. Subsequently, the EXPOSE mouse- and rabbit-specific horseradish peroxidase-diaminobenzidine detection immunohistochemistry kit (Abcam) was used. Finally, the sections were counterstained with hematoxylin. The data were quantified using ImageJ software by defining the immunostaining of positive cells.

### Embodiment 10: Chondrocyte Exposure to the Rhomboid Protease RHBDL2 Liberates Soluble TM (sTM), Inducing Cell Proliferation and Migration

Previous studies have indicated that the levels of soluble TM in the synovial fluid of patients with rheumatoid arthritis are elevated. However, there is currently no direct evidence to support a connection between TM expression and chondrocyte function and integrity. Therefore, in the present invention, chondrocytes were first tested whether they expressed TM or released sTM. The human chondrocyte cell line, TC28a2, was cultured to confluence under serum-free conditions.

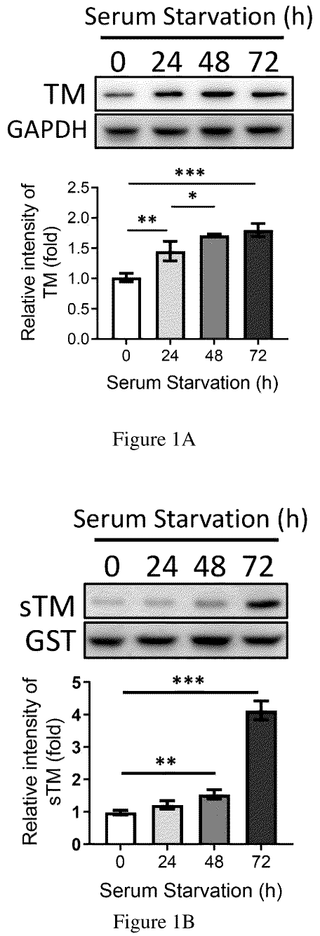

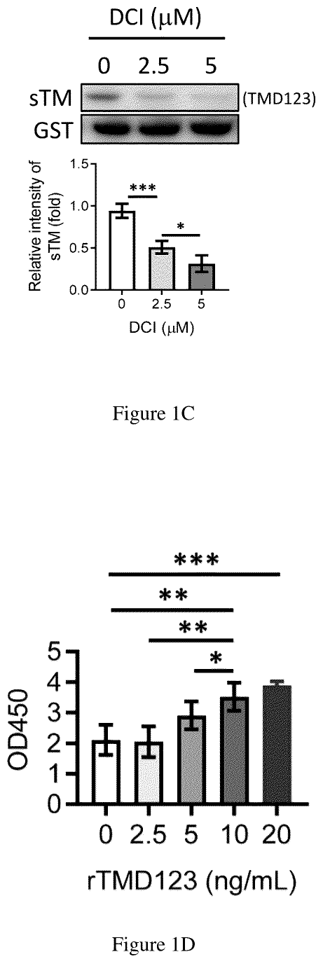

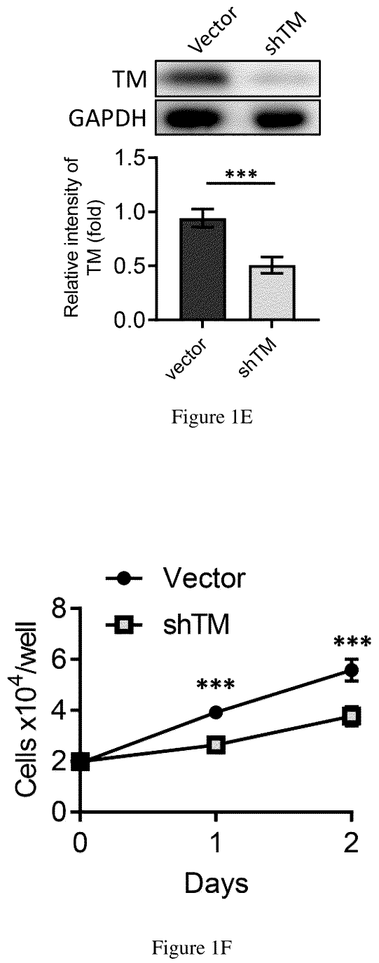

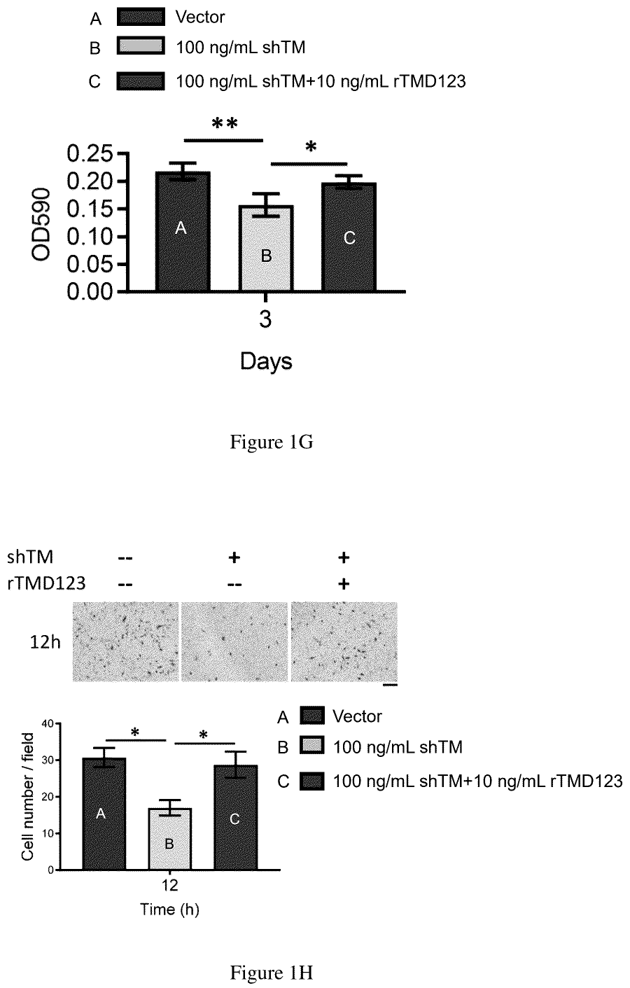

Cell lysates and conditioned media (CM) were collected daily for 72 h for western blotting to evaluate TM and sTM expression. Under these conditions, as shown in FIG. 1A, chondrocyte TM steadily increased over time. And as shown in FIG. 1B, a significantly increase in sTM production was detected in CM after 72 h. As shown in FIG. 1C, when cells were co-incubated with a specific inhibitor of RHBDL2 (DCI), the release of sTM reduced in a dose-dependent manner. In the present invention, the sTM contained the TM extracellular domain (domains 1 to 3, TMD123) because the cutting site of RHBDL2 was TMD4.

Induction of chondrocyte proliferation and migration may promote the healing of osteochondral defects. In this embodiment, as shown in FIG. 1D, in experiments in which 0, 2.5, 5, 10, and 20 ng/mL exogenous recombinant TMD123 (rTMD123) was added to chondrocytes, rTMD123 enhanced chondrocyte proliferation in a dose-dependent manner. As shown in FIG. 1G-H, the effects of TM on cell growth and migration were abrogated by the addition of 100 ng/mL TM-specific shRNA (shTM), which significantly reduced TM protein levels (FIG. 1E) and cell growth (FIG. 1F). Therefore, it is shown from this embodiment that chondrocytes produce full-length and soluble TM forms that may participate in cellular proliferation and migration.

### Embodiment 11: RTMD123 Inhibits STAT3/MMP 13 Signaling and IL-1β-Mediated Suppression of TM Expression in Chondrocytes

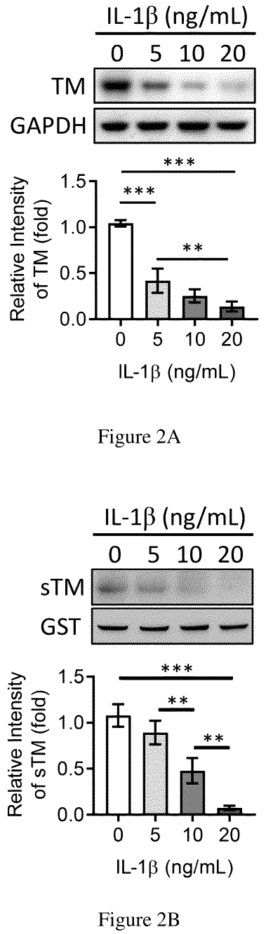

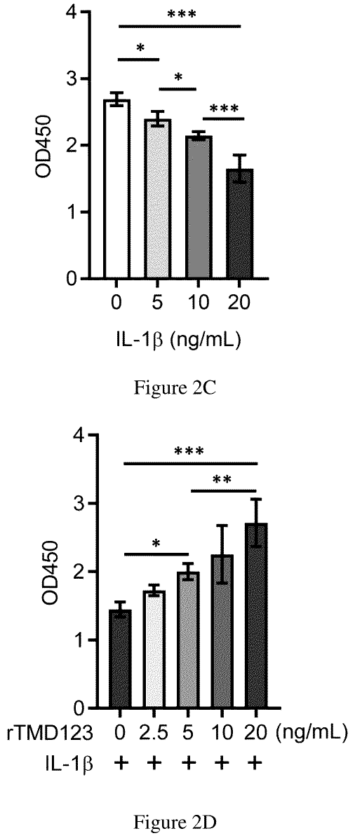

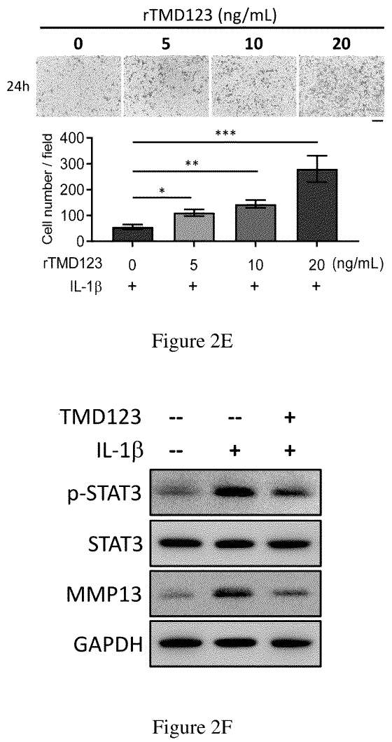

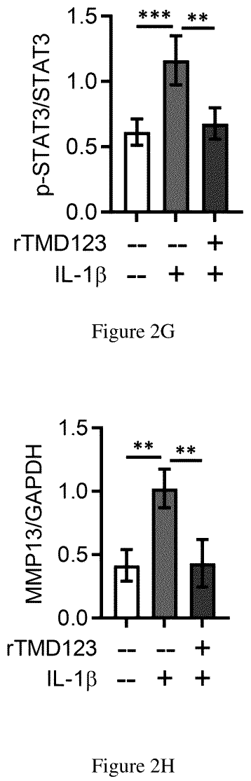

Proinflammatory cytokines, including interleukin (IL)-1β, tumor necrosis factor α (TNFα), IL-6, IL-15, IL-17, and IL-18, and IL-6/STAT3/MMP 13 signaling have been implicated in OA progression. This embodiment tested whether chondrocyte TM is regulated by IL-1β and whether such a relationship may be relevant in chondrocyte function. As shown in FIG. 2A-2C, in vitro experiments revealed that IL-1β decreased TM protein levels, sTM release, and cell proliferation. However, the dampening effects of IL-1β on cell proliferation and migration were abrogated by rTMD123. Even in the presence of 10 ng/ml IL-1β, 5-20 ng/mL rTMD123 promoted cell proliferation and migration and inhibited IL-1β-enhanced STAT3/MMP13 signaling, as shown in FIG. 2D-H.

### Embodiment 12: RTMD123 Protects Mouse Knees from ACLT-Induced OA and Dysfunction, Increases Articular Cartilage TM and Reduces MMP 13

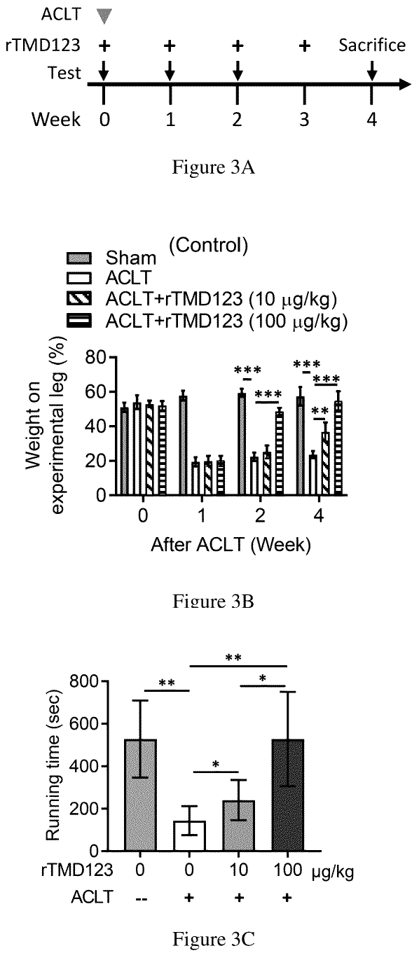

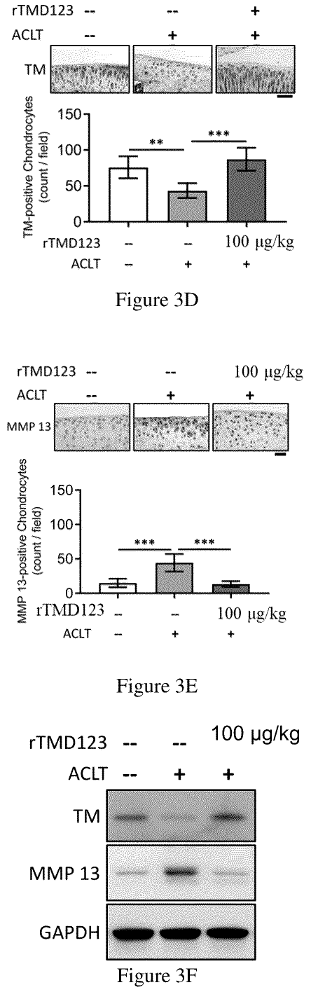

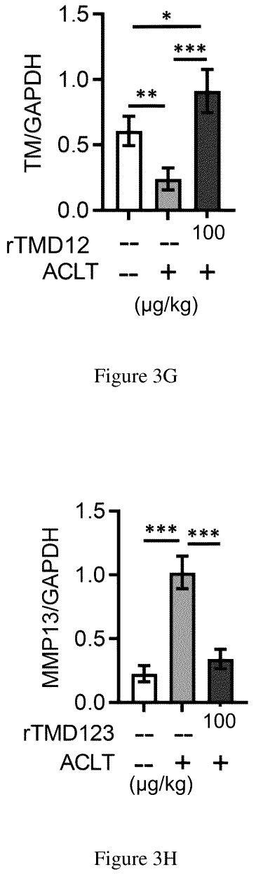

To evaluate the in vivo effects of TM on chondrocytes and joint function, this embodiment used the well-established mouse model of ACLT-induced OA. As shown in 3 A, 10 μg/kg or 100 μg/kg rTMD123 was injected into the knee joints of the mice once a week after ACLT surgery. As shown in FIG. 3B, rTMD123 injection significantly counteracted the reduced weight-bearing capacity of ACLT in a dose-dependent manner, with the maximal benefit achieved at four weeks. As shown in FIG. 3C, similar beneficial results were evident in the running test. Immunohistochemical (IHC) staining and western blotting of articular cartilage showed that rTMD123 treatment-maintained TM levels and inhibited MMP13 expression levels in the presence of OA, as shown in FIG. 3D-3H. Furthermore, the TM protein level was also dramatically reduced in the articular cartilage sections of patients with OA. Therefore, TM and sTM have potential roles in protecting chondrocytes from pathological changes associated with OA.

### Embodiment 13: RTMD123 Rescues Knee Dysfunction and Chondro-Cartilage Joint Damage in TM^{LeD/LeD} Mice with OA

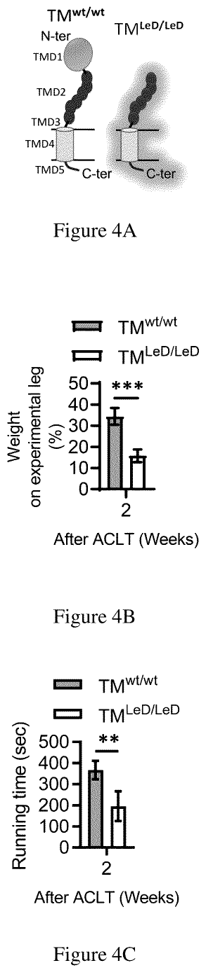

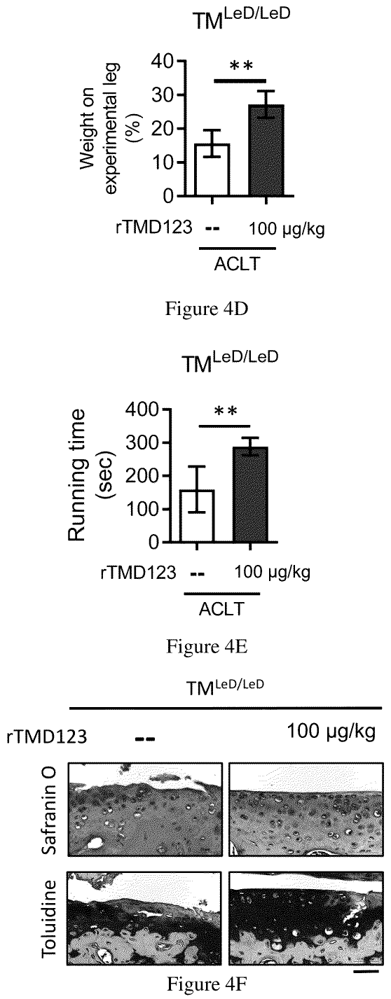

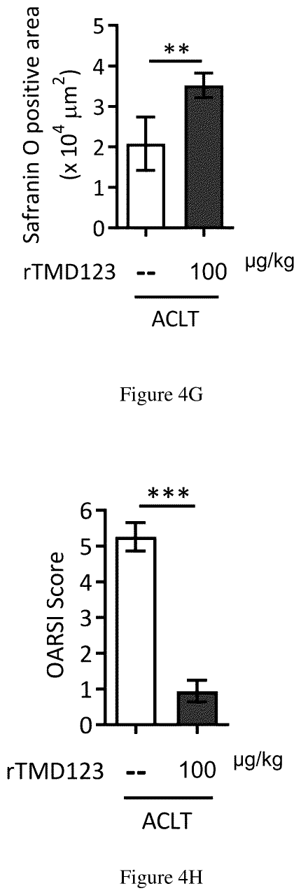

In different OA models, inflammatory arthritis develops more rapidly and severely in TM^{LeD/LeD} mice than in wild-type (TM^{wt/wt}) mice. There is no significant difference in appearance between TM^{LeD/LeD} mice and TM^{wt/wt} mice. However, after ACLT surgery 2 weeks, functional tests on the knee joint showed a significant loss of knee function in TM^{LeD/LeD} mice compared to that in TM^{wt/wt} mice, as shown in FIG. 4B-C. Notably, articular joint injection of 100 μg/kg rTMD123 can protect TM^{LeD/LeD} mice from knee dysfunction while also preventing the loss of articular cartilage, as shown in FIG. 4D-4G. These results further confirm the importance of TM, the lectin-like domain of TM, and TMD123 in models of acute inflammatory arthritis and OA and chondrocyte function and integrity.

### Embodiment 14: RTMD123 Protects Against Knee Dysfunction and Articular Cartilage Loss after OA-Injury Induction

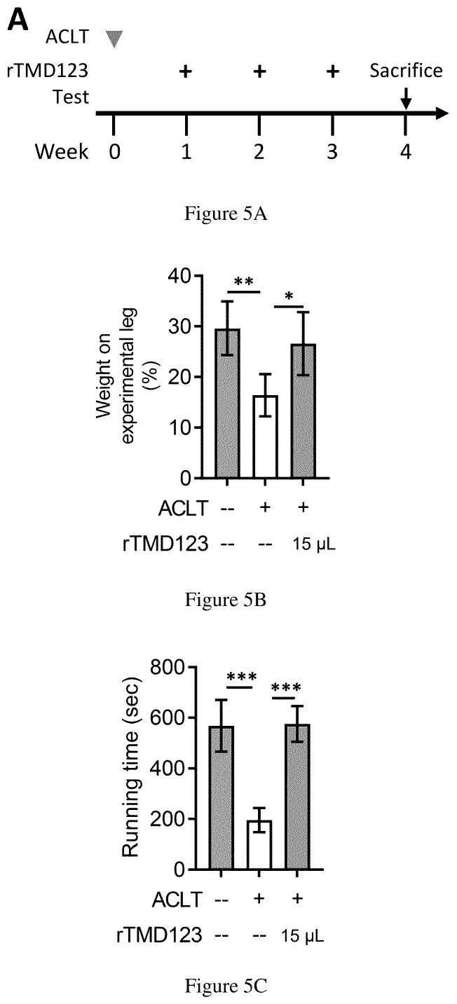

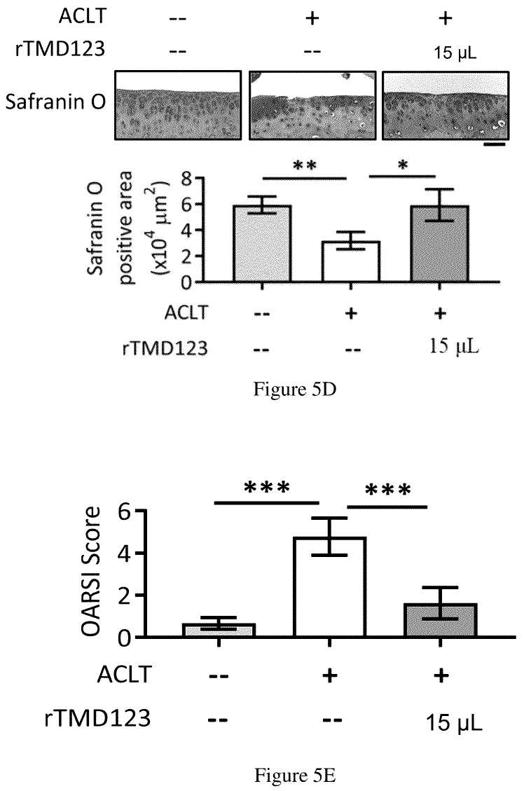

To better represent the clinical situation, we assessed the efficacy of administering rTMD123 after joint damage was induced using the ACLT-OA model. As shown in the test process in FIG. 5A, when mice began to receive weekly injections of 5 μL of rTMD123 into the knee joint one week after surgery, and a total of 15 μL was injected for three weeks, rTMD123 still protected joint function and cartilage integrity, as shown in FIG. 5B-5E.

### Embodiment 15: TM Silencing Inhibits miR-150 Antagomir (miR-Up-TM)-Increased the Expression of KLF2/TM and Abolishes the TM-Mediated Protective Effects on Knee Dysfunction in the OA Model

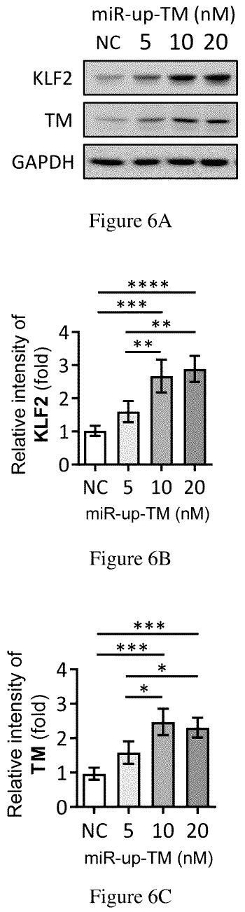

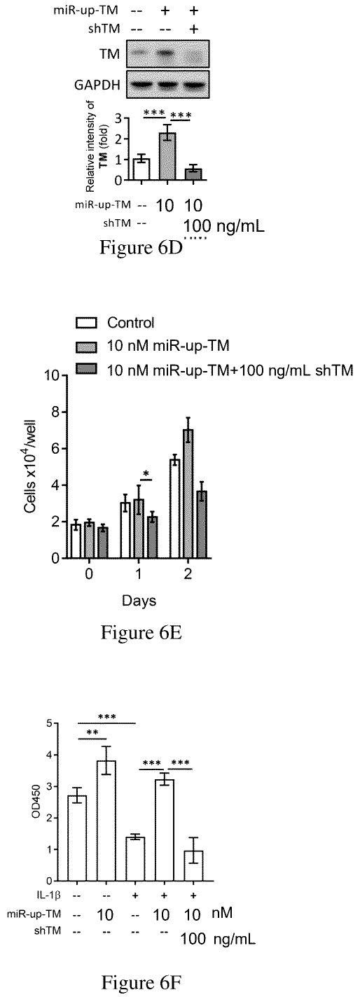

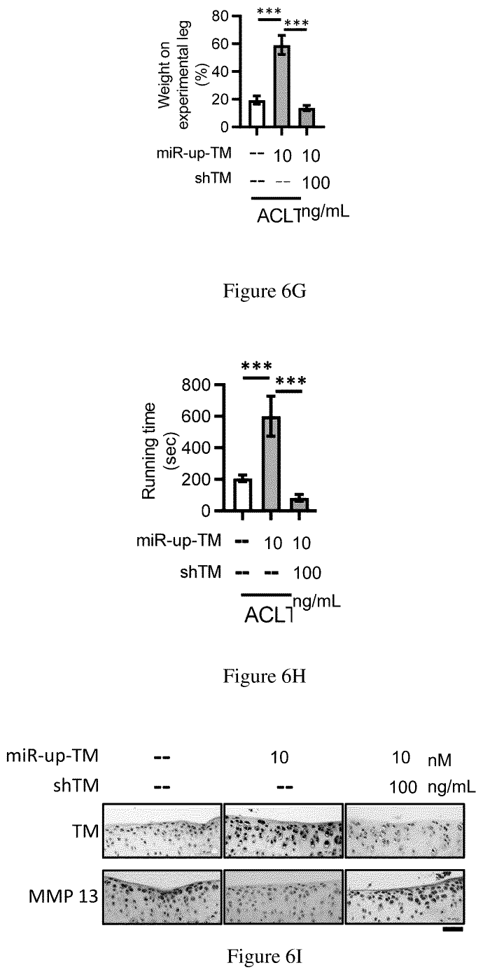

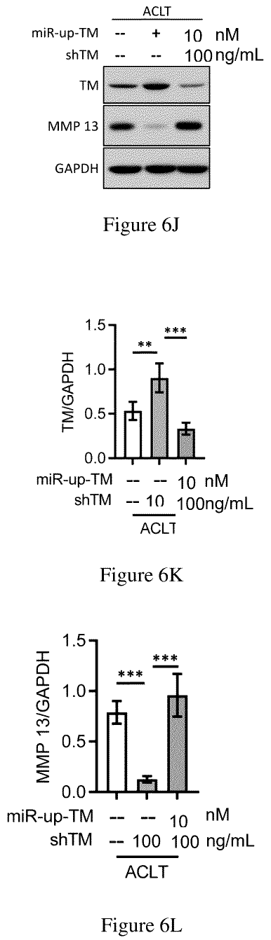

A specific miRNA inhibitor enhances the expression of KLF2/TM in human endothelial cells; however, its effects on chondrocytes or OA remain unclear. Therefore, in this embodiment, in vitro test showed that miR-up-TM dose-dependently enhanced the expression of KLF2 and TM in chondrocytes, as shown in FIG. 6A-6C, and this effect was associated with increased cell proliferation and interference with IL-1β-suppressed cell growth, as shown in FIG. 6E-6F. As shown in FIG. 6D-6F, the miR-up-TM-enhanced TM levels and their associated benefits were significantly reduced by shTM. As shown in FIG. 6G-6H, in line with our in vitro findings, the administration of 10 nM miR-up-TM to mice following ACLT surgery yielded similar protective effects, with reduced knee dysfunction and cartilage loss. As shown in FIG. 6G-6L, these beneficial responses were abolished by shTM, which caused TM silencing and increased MMP 13.

The RNA nucleotide sequence of the miRNA inhibitor used in the present invention is UCUCCCAACCCUUGUACCAGUG (SEQ ID NO:1).

### Embodiment 16: The Mechanism of rTMD123 Affecting Chondrocytes

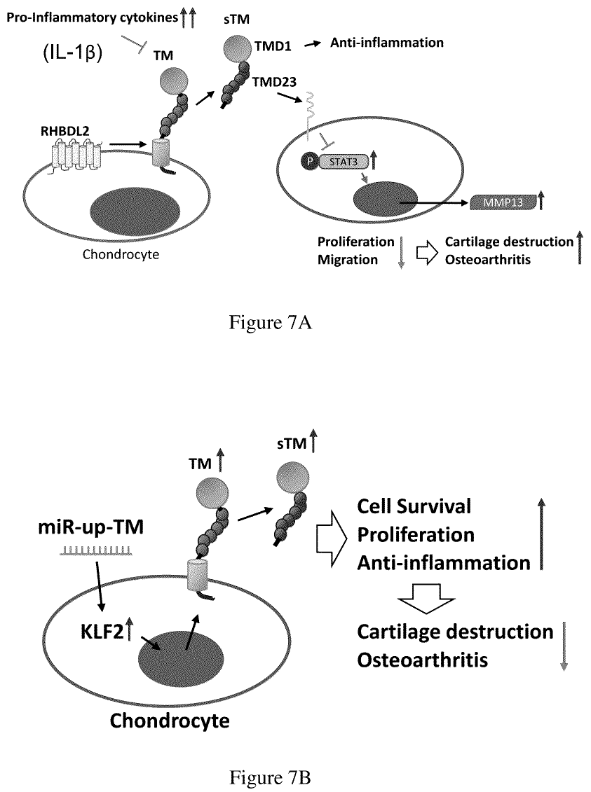

The present invention reveals the relationship between pro-inflammatory cytokines (IL-1β) and OA. IL-1β inhibits the expression of TM in chondrocytes, leading to increased STAT3/MMP13 signaling and the formation of OA. However, as shown in FIG. 7A, treatment with rTMD123 significantly attenuated the effects mediated by IL-1β. TM may be a pivotal blocker of IL-1β-suppressed cell growth and cartilage loss in chondrocytes. Therefore, TM and rTMD123 are potential novel agents for the treatment and prevention of OA. An additional intriguing approach to modulating the expression of TM in OA was further revealed by the present invention with the miRNA-150 antagonist, as shown in FIG. 7B, which enhanced the expression of KLF2 and TM and interfered with OA progression to combat diseases related to chondrocyte damage.

What is claimed is:

1. A method for prevention or treatment of a cartilage-related disorder in a subject, comprising administering to the subject an effective amount of a composition comprising Thrombomodulin.

2. The method of claim 1, wherein the composition further comprises a pharmaceutically acceptable carrier comprising plasma, optionally enriched with platelets, serum, water for injection, physiological saline, hyaluronan, chemically modified hyaluronan, saline, phosphate buffered saline, chondroitin sulfate, glucosamine, mannosamine, proteoglycan, proteoglycan fragments, chitin, chitosan, or a combination of two or more thereof.

3. The method of claim 1, wherein the cartilage-related disorder is osteoarthritis, degenerative joint disease, osteochondritis dissecans, rheumatoid arthritis, articular cartilage damage, achondroplasia, or cartilage defects.

4. The method of claim 1, wherein the composition reduces cellular inflammatory response, promotes cell growth and migration, increases the expression of Krüppel-Like Factor 2 (KLF2) and thrombomodulin, blocks interleukin-1β (IL-1β)-mediated signal transduction, protects knee joints, prevents cartilage loss or damage, maintains the integrity of bone joints, and preserves the subject's mobility by administering the effective amount to the subject, thereby preventing or treating cartilage-related disorders.

5. The method of claim 4, wherein the IL-1β-mediated signaling comprises STAT3 and MMP13.

6. The method of claim 1, wherein Thrombomodulin is natural or synthetic protein.

7. The method of claim 1, wherein the effective amount of Thrombomodulin is 1˜100 μg/kg.

8. A method for prevention or treatment of a cartilage-related disorder in a subject, comprising administering to the subject an effective amount of a composition comprising miR-up-TM.

9. The method of claim 8, wherein the composition further comprises a pharmaceutically acceptable carrier comprising plasma, optionally enriched with platelets, serum, water for injection, physiological saline, hyaluronan, chemically modified hyaluronan, saline, phosphate buffered saline, chondroitin sulfate, glucosamine, mannosamine, proteoglycan, proteoglycan fragments, chitin, chitosan, or a combination of two or more thereof.

10. The method of claim 8, wherein the cartilage-related disorder is osteoarthritis, degenerative joint disease, osteochondritis dissecans, rheumatoid arthritis, articular cartilage damage, achondroplasia, or cartilage defects.

11. The method of claim 8, wherein the composition reduces cellular inflammatory response, promotes cell growth and migration, increases the expression of Krüppel-Like Factor 2 (KLF2) and thrombomodulin, blocks interleukin-1β (IL-1β)-mediated signal transduction, protects knee joints, prevents cartilage loss or damage, maintains the integrity of bone joints, and preserves the subject's mobility by administering the effective amount to the subject, thereby preventing or treating cartilage-related disorders.

12. The method of claim 8, wherein the miR-up-TM is miR-150 antogomir.

13. The method of claim 8, wherein the miR-150 antogomir is RNA sequence of SEQ ID NO:1.

14. The method of claim 8, wherein the effective amount of the miR-up-TM is 0.1˜20 nM.

15. The method of claim 11, wherein the IL-1β-mediated signaling comprises STAT3 and MMP13.
